(19) United States
(12) Patent Application Publication
(10) Pub. No.: US 2025/0243140 A1
(43) Pub. Date: Jul. 31, 2025

(54) NUCLEAR PROCESS STEAM DRIVEN HYDROTHERMAL DECOMPOSITION OF METHANE FOR LOW-TEMPERATURE GREEN METHANOL PRODUCTION

(71) Applicant: NuScale Power, LLC, Corvallis, OR (US)

(72) Inventors: Francis Y. Tsang, Bellingham, WA (US); José N. Reyes, Jr., Corvallis, OR (US); Luis DePavia, Corvallis, OR (US)

(73) Assignee: NuScale Power, LLC, Corvallis, OR (US)

(21) Appl. No.: 19/036,814

(22) Filed: Jan. 24, 2025

Related U.S. Application Data
(60) Provisional application No. 63/625,621, filed on Jan. 26, 2024.
(60) Provisional application No. 63/572,962, filed on Apr. 2, 2024.

(51) Int. Cl. C07C 29/152; B01J 19/24; C25B 1/23; C25B 9/77; C25B 15/08

(52) U.S. Cl. CPC C07C 29/152; B01J 19/245; C25B 1/23; C25B 9/77; C25B 15/081

(57) ABSTRACT

An integrated energy system including a power plant is discussed herein. In some examples, the integrated energy system may include a power plant configured to generate steam, a hydrothermal decomposition reactor configured to receive at least a portion of the steam (H₂O) from the power plant to react with Methane (CH₄) within the hydrothermal decomposition reactor to produce Hydrogen (H₂) and Carbon Dioxide (CO₂), a first separation unit configured to separate the Hydrogen (H₂) and the Carbon Dioxide (CO₂), a Solid Oxide Stack configured to receive at least a portion of the Carbon Dioxide (CO₂) and to produce Carbon Monoxide (CO), a second separation unit configured to separate the Carbon Dioxide (CO₂) from the Carbon Monoxide (CO), and a methanol synthesis reactor configured to receive at least a portion of the Hydrogen (H₂) and at least a portion of the Carbon Monoxide (CO) to produce Methanol (CH₃OH).

### CROSS-REFERENCE TO RELATED APPLICATIONS

This application claims the benefit of U.S. Provisional Patent Application No. 63/625,621, filed Jan. 26, 2024, entitled “NUCLEAR DRIVEN STEAM-METHANE REFORMING PROCESS FOR GREEN METHANOL PRODUCTION”, and 63/572,962, filed Apr. 2, 2024, entitled “NUCLEAR DRIVEN STEAM-METHANE REFORMING PROCESS FOR GREEN METHANOL PRODUCTION” which are incorporated herein by reference in their entirety.

### TECHNICAL FIELD

The present technology is directed to nuclear reactor integrated energy systems (IESs) for energy production and green industrial applications, such as to IESs including one or more nuclear reactors (e.g., small modular nuclear reactors (SMRs)) coupled to a Hydrothermal Decomposition of Methane (HTDM) process for producing hydrogen gas and carbon dioxide gas for methanol production, and associated devices and methods.

### BACKGROUND

The energy production landscape has evolved rapidly in recent years, with a growing emphasis on decarbonization, sustainability, and resilience, driving the adoption of cleaner and more efficient forms of power production. While fossil fuels continue to play a significant role in global energy supply, there is a need for increased deployment of renewable energy, coupled with advancements in energy storage, grid modernization, and energy efficiency measures, to address the challenges of climate change and energy transition.

Cumulative carbon dioxide emissions are the dominant driver of climate change. The largest carbon emission sources are attributed to the hydrocarbon fuel combustion in electricity, heat generation, and chemical industry which contributes to nearly two-thirds of the total worldwide CO₂ emissions. The seven largest CO₂ emission industries in the world are: (1) power plants (coal, natural gas, oil fired), (2) oil refinery plants, (3) ammonia production plants, (4) chemical manufacturing and production plants, (5) cement production plants, (6) steel manufacturing plants, and (7) transportation. The International Energy Agency (IEA) reported that the CO₂ emission from industrial processes and energy combustion grew by 321 Mt in 2022.

Many of these processes, as well as others in the petroleum, chemical, pharmaceutical, and material manufacturing industries require a combination of electrical power, steam, heat, and Hydrogen (H₂) to operate and to produce industrial products. For example, Hydrogen (H₂) is used in each of the above industries (2)-(7). Currently, most of the Hydrogen (H₂) produced in the United States comes from steam-methane reforming processes. In the United States, steam-methane reforming processes accounted for more than 95% of all Hydrogen (H₂) production and produced about 10 million metric tons (MT) of H₂ each year. Nearly 70% of this hydrogen is used in the petroleum refining industry, and 20% is used for fertilizer production. The remaining 10% is used in chemical and material production processes. Typically, approximately 0.71 metric tons of CO₂ equivalent are emitted per metric ton of methanol produced when producing methanol using steam-methane reforming making it a significant contributor to greenhouse gas emissions.

An energy system incorporates various energy conversion technologies, such as power plants, cogeneration (combined heat and power) systems, and distributed generation units (such as solar panels and wind turbines). These technologies convert primary energy sources into usable forms of energy, such as electricity, heat, steam, and mechanical power that can be used as secondary energy sources. Energy systems leverage a diverse range of energy resources, including renewable energy sources (such as solar, wind, and hydroelectric power), conventional fuels (such as natural gas and coal), and emerging technologies (such as hydrogen and biofuels). By combining multiple energy sources, these systems can enhance energy security and resilience.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures may indicate similar or identical items. Furthermore, the drawings may be considered as providing an approximate depiction of the relative sizes of the individual components within individual figures. However, the drawings are not to scale, and the relative sizes of the individual components, both within individual figures and between the different figures, may vary from what is depicted. In particular, some of the figures may depict components as a certain size or shape, while other figures may depict the same components on a larger scale or differently shaped for the sake of clarity.

FIG. 1 is a schematic diagram of an integrated energy system for producing Methanol that includes a power plant system in accordance with at least some embodiments of the present disclosure.

FIG. 2 is a schematic diagram of an integrated energy system for producing Methanol that includes a power plant system in accordance with embodiments of the present disclosure.

FIG. 3 is a block diagram illustrating an example Hydrothermal Decomposition Plant for producing Hydrogen and Carbon Dioxide in accordance with embodiments of the present disclosure.

FIG. 4 is a block diagram illustrating the utilization of a Solid Oxide Stack, in accordance with embodiments of the present disclosure.

FIG. 5 depicts a block diagram illustrating an example Methanol Production Plant for producing Methanol in accordance with embodiments of the present disclosure.

FIG. 6 is a schematic view of a nuclear power plant system including multiple nuclear reactors in accordance with embodiments of the present technology.

FIG. 7 is a partially schematic, partially cross-sectional view of a nuclear reactor system configured in accordance with embodiments of the present technology.

FIG. 8 is a partially schematic, partially cross-sectional view of a nuclear reactor system configured in accordance with embodiments of the present technology.

FIG. 9 illustrates an example process of the hydrothermal decomposition of methane to produce hydrogen and carbon dioxide for methanol production, in accordance with embodiments of the present disclosure.

### DETAILED DESCRIPTION

In embodiments, the present disclosure is directed to techniques that may be performed in relation to Integrated Energy Systems (IESs), such as for use in green industrial processes that produce few or no carbon emissions, to capture carbon from an emission source, for resource production, and associated devices and methods. IESs of the present technology may include a power plant (e.g., a primary power plant) that is integrated with one or more industrial processes and resource production plants to provide power with few or no carbon emissions using excess power and/or steam from the power plant. The present disclosure includes systems and methods that may address many problems associated with conventional resource production processes, such as reducing carbon emissions and improving economic viability.

Because of the drive toward cleaner and more efficient forms of power production, nuclear power will be increasingly important in the coming years. In operation, nuclear power plants use the nuclear fission process to generate heat, which is then used to produce steam to turn turbines and generate electricity. This process can result in the production of electrical power that reduces the need for coal and natural gas to produce electricity. Nuclear power plants provide reliable baseload power without emitting greenhouse gases such as Carbon Dioxide (CO₂) during operation, making them attractive for countries that are seeking to reduce carbon emissions and enhance energy security. Due to the advantages of nuclear energy for providing electricity, the present disclosure presents novel methods of using nuclear power in integrated energy systems for carbon capture and “green” resource production, such as the production of “green” chemical products.

In embodiments, the IES includes a power plant system having multiple small modular nuclear reactors (SMRs) specifically configured to operate in unison to support one or more of the industrial processes. SMRs are nuclear reactors that are smaller in terms of size (e.g., dimensions) and power compared to large, conventional nuclear reactors. Moreover, they are modular in that some or all of their systems and components can be factory-assembled and transported as a unit to a location for installation. In some aspects of the present technology, the multiple SMRs of the integrated energy system can flexibly and dynamically provide electricity, thermal, steam, or a combination of electricity, thermal, and steam to the industrial processes due to the modularity and flexibility of the SMRs. That is, a configuration of the SMRs can be switched during operation to provide varying levels of steam and electricity output depending on the operational states and/or demands of the industrial processes.

In embodiments, a power plant of the present disclosure can be a permanent or temporary installation built at or near (e.g., roughly 1 km from) the location of an industrial process facility or can be a mobile or partially mobile system that is moved to and assembled at or near the location of the industrial process facility. More generally, the power plant can be local (e.g., positioned at or near) to the industrial processes/operations it supports. For example, the power plant can be located within 0.4 km (0.25 mile), within 0.8 km (0.5 mile), within 3.22 km (2 miles), within 4.82 km (3 miles), or within 8.1 km (5 miles) of the industrial processes/operations it supports. In embodiments, the power plant is configured to supply a portion of electricity to a power grid.

For example, an IES of the present disclosure may include a Hydrothermal Decomposition of Methane (HTDM) process for resource production and associated devices and methods. Resource production may include producing and purifying hydrogen gas (H₂) and carbon dioxide gas (CO₂) in a HTDM plant, producing and purifying carbon monoxide gas (CO) and oxygen gas (O₂) in an electrolysis plant, and producing methanol (CH₃OH) in a methanol synthesis reactor. In embodiments, the hydrogen (H₂) from the HTDM plant and the carbon monoxide (CO) from the electrolysis plant may be used to produce methanol (CH₃OH) in the methanol synthesis reactor. In embodiments, the Carbon Dioxide (CO₂) from the HTDM plant may be used to produce the Carbon Monoxide (CO) and the Oxygen (O₂) in the electrolysis plant. In embodiments, resources produced in an IES of the present disclosure may be recycled within the IES, removed from the IES, stored for future use, or sold.

Methanol (CH₃OH) is a highly versatile chemical widely used for industrial purposes and prevalent in our everyday lives, additionally it is an energy-intensive source and sustainable fuel that could help in shaping the future economy. Methanol is a base material in the production of acetic acid and formaldehyde, and also increasingly being used in ethylene and propylene production. Methanol is one of the most prolific intermediate materials for the production of other chemicals and materials. In the chemical industry methanol mainly serves as a raw material in the production of formaldehyde, olefins, acetic acid, MTBE, DME, as well as biodiesel. So, renewable methanol is a pre-requisite for making a broad range of chemical products green such as polymer fibers for the textile industry, plastics for packaging, glues, adsorbents/diapers, paints, adhesives, solvents, and much more. While Methanol (CH₃OH) is a vital chemical, it is also extremely corrosive and therefore must be handled with extreme caution. For example, proper engineering controls must be designed and used to maintain safe manufacture, storage, and handling of the material.

Besides its use in the chemical, construction and plastics industries, methanol also serves as a fuel or fuel additive. Methanol is an extremely efficient hydrogen carrier because one methanol molecule has more hydrogen atoms than one hydrogen molecule. Methanol is a liquid at ambient conditions. Therefore, methanol can be handled, stored, and transported with ease by leveraging existing industrial infrastructures. The global methanol demand reached 98 million tons in 2019 and is expected to rise to approximately 500 million tons by 2050. This implies that more sustainable methanol manufacturing processes are needed in the near future. In addition, considering the global climate challenges, methanol is a potential alternative fuel to scale down the reliance on fossil fuels as a means of energy storage and transportation.

The conventional production method for Methanol (CH₃OH) involves a catalytic process using fossil fuel feedstock such as a steam-methane-reforming process with natural gas or a coal gasification process. About 90% of methanol production is currently from natural gas, and other technologies cannot be substituted on an industrial scale due to undesirable efficiency. Currently, synthesis gas (syngas), a mixture of Hydrogen (H₂), Carbon Monoxide (CO), and Carbon Dioxide (CO₂), is the primary feedstock for methanol production, and natural gas is used to supply Methane (CH₄) for syngas production. Currently, there is no existing process that can simultaneously produce all necessary gases for Methanol (CH₃OH) production and the required energy demand with few or no carbon emissions.

The conventional production of methanol from syngas can be summarized into two basic steps: (1) production of syngas, and (2) syngas conversion to methanol. The predominant method of producing syngas for methanol production is through the hydrothermal decomposition of Methane (CH₄) with high-temperature steam (H₂O), i.e., steam-methane reforming. In steam-methane reforming, Methane (CH₄) reacts with steam (H₂O) following the reaction below in Equation 1 to produce Hydrogen (H₂) and Carbon Monoxide (CO):

$${CH}_{4} + {H}_{2} O ↔ CO + 3 {H}_{2} \qquad ( 1 )$$

The produced Carbon Monoxide (CO) and steam (H₂O) also react to produce Carbon Dioxide (CO₂) in the water gas shift (WGS) reaction shown below in Equation 2:

$$CO + {H}_{2} O ↔ {CO}_{2} + {H}_{2} \qquad ( 2 )$$

Combining these two Equations, the complete steam-methane reforming reaction can be expressed as Equation 3:

$$C {H}_{4} + 2 {H}_{2} O ↔ {CO}_{2} + 4 {H}_{2} \qquad ( 3 )$$

Conventional steam-methane reforming is a highly endothermic process in which the operating temperature is in the range of about 700-1000° C., the pressure is about 1-50 bar and the reaction may occur in the presence of a catalyst, for example, a group 8 to 10 metal-based catalyst such as a nickel (Ni) based catalyst. Steam-methane reforming therefore requires large energy input. Presently, fossil fuels such as natural gas, petroleum, and coal must be used to supply the required reformer heat demand. The current steam-reforming process, therefore, results in significant Carbon Dioxide (CO₂) emissions into the atmosphere which is a concern for its environmental impact pertaining to green-house gas concentrations affecting global climate change. Environmental analysis shows that approximately 0.71 metric tons of CO₂ equivalent are emitted per metric ton of methanol produced when producing methanol using steam methane reforming.

While syngas is mainly derived from natural gas by steam-methane reforming, there are several other pathways to producing syngas depending on feedstock, oxidation agent, desired syngas ratio, and downstream application. For example, syngas can also be generated by partial oxidation (PO) of methane, petroleum, coke, heavy oils, or coal. When natural gas is the feedstock, more than half of the total expenditure of materials, energy, and cost is required with the production process.

An IESs of the present disclosure can reduce and/or eliminate carbon emissions associated with typical syngas production via steam-methane reforming by coupling low-temperature hydrothermal decomposition of Methane (CH₄) to an emission free energy source, such as one or more small modular nuclear reactor (SMR) described herein. The low temperature hydrothermal decomposition of Methane (CH₄) provides advantages over traditional high-temperature steam-methane reforming. The low-temperature process requires less stringent equipment and operational control, which in a long run, results in lower operational costs and the equipment would be less expensive and will last much longer than current practices. In addition, replacement cost of catalysts due to de-activation at much higher temperature also could have substantial cost saving.

Not only can a small modular nuclear reactor (SMR) of the present disclosure produce emission free energy, but it can also generate super-heated process steam for industrial applications, see for example, Applicant's U.S. patent application Ser. No. 18/116,819, filed on Mar. 2, 2023, and entitled “Small Modular Nuclear Reactor Integrated Energy Systems for Energy Production and Green Industrial Applications” which is incorporated herein by reference in its entirety. An IES of the present disclosure therefore can suppress CO₂ emission during the steam-methane reforming process, eliminate the use of natural gas an as energy source to support the entire production cycle, and provide super-heated process steam, such as super-heated steam, for the hydrothermal decomposition of Methane (CH₄).

Currently, most Methanol (CH₃OH) is produced by hydrogenating Carbon Dioxide (CO₂) with Hydrogen (H₂). Many studies in the past have explored different aspects of Carbon Dioxide (CO₂) hydrogenation for methanol production primarily due to an increased focus on carbon emissions reduction under the carbon utilization strategy. So far, none can prove or provide concrete evidence to reduce carbon dioxide emission pertaining to the production of Methanol via the Steam-Methane Reforming process. Carbon Dioxide (CO₂) hydrogenation reaction conditions include high pressure (50-100 bar) and moderate temperatures (about 200-300° C., or around 250° C.). the reaction proceeds with high selectivity on a metal catalyst, such as a conventional Copper (Cu) and zinc oxide (ZnO) based catalyst (Cu/ZnO), following the reaction in Equation 4:

$${CO}_{2} + {3 H}_{2} {↔}^{Cu / ZnO} {CH}_{3} O H + {H}_{2} O \qquad ( 4 )$$

For example, syngas produced through steam-methane reforming is typically fed to a methanol synthesis reactor the production of Methanol (CH₃OH). As shown in Equation 1, the produced syngas contains four moles of hydrogen for every mole of carbon dioxide. Typically, Carbon Dioxide (CO₂) is injected into the methanol synthesis reactor to react with the extra hydrogen to complete the Methanol production process, shown in Equation 4.

The synthesis of Methanol (CH₃OH) from Carbon Dioxide (CO₂) is complicated, however, because of water formation. In the absence of Carbon Monoxide (CO), water is produced as the by-product of Carbon Dioxide (CO₂) hydrogenation. The increased formation of water leads to kinetic inhibition and accelerate deactivation of the necessary catalysts, such as Cu/ZnO catalysts. To remove water and prevent the deactivation of the catalyst, Carbon Monoxide (CO) can be injected into the reaction chamber to react with water via the Water-Gas-Shift (WGS) reaction in Equation 2, shown again below:

$$CO + {H}_{2} O ↔ {CO}_{2} + {H}_{2} \qquad ( 2 )$$

While, the introduction of Carbon Monoxide (CO) can remove water produced in Equation 4 via the Water-Gas-Shift (WGS) reaction, it may not eliminate water formation. Liquid water can accelerate deactivation by speeding up the growth of copper crystals and destroying the catalyst matrix.

Methanol (CH₃OH) may also be produced industrially by hydrogenation of Carbon Monoxide (CO) over a catalyst. The most widely used catalyst is a mixture of Copper and Zinc oxides at temperature at about 250° C. and between 5-10 MPa (50-100 atm). The reaction is shown in Equation 5:

$$CO + 2 {H}_{2} {↔}^{Cu / ZnO} {CH}_{3} O H \qquad ( 5 )$$

The hydrogenation of Carbon Monoxide (CO) for Methanol (CH₃OH) production is not common in industry, however, due to the cost, energy demands, and environmental impacts of Carbon Monoxide (CO) production using conventional methods. One unique advantage of Integrated Energy Systems (IESs) of the present disclosure is the ability to reduce the cost, energy demand, and environmental impact of Carbon Monoxide (CO) production.

Another unique aspect of IESs of the present disclosure is the utilization of an electrolysis process, for example a solid oxide stack, such as a Solid Oxide Electrolysis Cell (SOEC) or a CO-Electrolysis Cell, to produce a combination of Carbon Monoxide (CO) and Carbon Dioxide (CO₂) gases for the production of “Green” Methanol without the use of another reforming processes to produce Carbon Monoxide (CO), see for example Applicant's U.S. patent application Ser. No. 18/486,971, filed on Oct. 13, 2023, and entitled “Small Modular Nuclear Reactor Integrated Energy Systems for In-Situ, On-Demand Hydrogen Generation and/or the Production of Sodium Formate,” which is incorporated herein by reference in its entirety. This setup eliminates additional carbon dioxide emission. related to a solid oxide stack for converting CO₂ to CO, CO₂, and O₂ is found under World patent WO 2014/154253 A1 “A process for producing CO from CO₂ in a solid oxide electrolysis cell.” This technology primarily utilizes a solid oxide electrolysis cell (SOEC) stack to achieve this conversion by applying an electric current to CO₂ on the fuel side, producing CO and releasing Oz on the oxygen side, and the disclosure is incorporated herein by reference in its entirety.

An IES of the present disclosure can improve current Methanol (CH₃OH) production by producing low carbon electrical and thermal energy for the production and purification of syngas into Carbon Dioxide (CO₂) and Hydrogen (H₂), for the conversion of Carbon Dioxide (CO₂) into Carbon Monoxide (CO), and for the production of Methanol (CH₃OH). In embodiments, an IES of the present disclosure can provide low carbon electrical and thermal energy to convert at least a portion of the purified Carbon Dioxide (CO₂) from the hydrothermal decomposition of methane (CH₄) to Carbon Monoxide (CO), for example in a solid oxide stack. In embodiments, purified Hydrogen (H₂) and Carbon Monoxide (CO) may then be fed to a methanol synthesis reactor to perform Carbon Monoxide (CO) hydrogenation at low temperatures compared to a Carbon Dioxide (CO₂) hydrogenation reaction, thus reducing the energy requirements. The hydrogenation of Carbon Monoxide (CO) to produce Methanol (CH₃OH) eliminates the formation of water, which prevents catalyst degradation and allows the use of more efficient catalysts, for example a Nickel Oxide (NiO₂). Note there are two moles of Hydrogen (H₂) per one mole of Carbon Monoxide (CO) in Equation 5, and an IES of the present disclosure can provide hydrogen in excess of two moles of Hydrogen (H₂) per one mole of Carbon Monoxide (CO) generated, for example, eight moles of Hydrogen (H₂) per one mole of Carbon Monoxide (CO). Therefore, excess hydrogen may be recycled within the IES, used in other processes connected to the IES, removed from the IES, stored for future use, and/or sold.

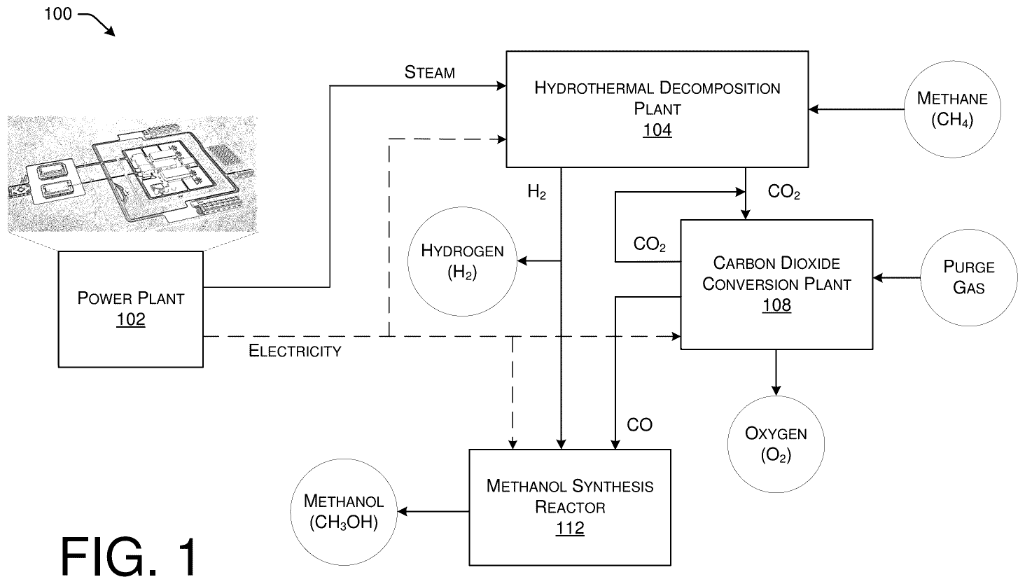

Certain details are set forth in the following description and in FIGS. 1-9 to provide a thorough understanding of various embodiments of the present technology. In other instances, well-known structures, materials, operations, and/or systems often associated with nuclear reactors, power plant systems, integrated energy systems, chemical production plants, industrial process plants, electrolysis systems, direct air capture (DAC) plants, oil refineries, and the like, are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. The terminology used below is to be interpreted in its broadest reasonable manner, even though it is being used in conjunction with a detailed description of certain examples of embodiments of the technology.

The accompanying Figures depict embodiments of the present technology and are not intended to limit its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the present technology. In addition, those of ordinary skill in the art will appreciate that further embodiments of the present technology can be practiced without several of the details described below.

Each of the references cited herein is incorporated herein by reference in its entirety. However, to the extent any materials incorporated herein by reference conflict with the present disclosure, the present disclosure controls. The headings provided herein are for convenience only and should not be construed as limiting the subject matter disclosed.

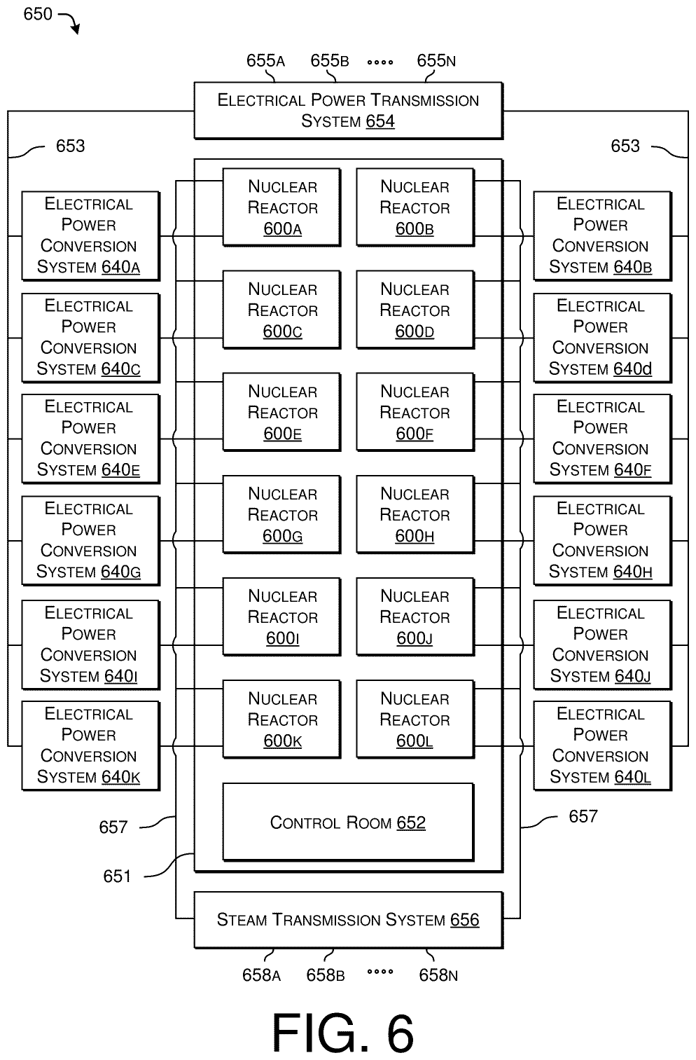

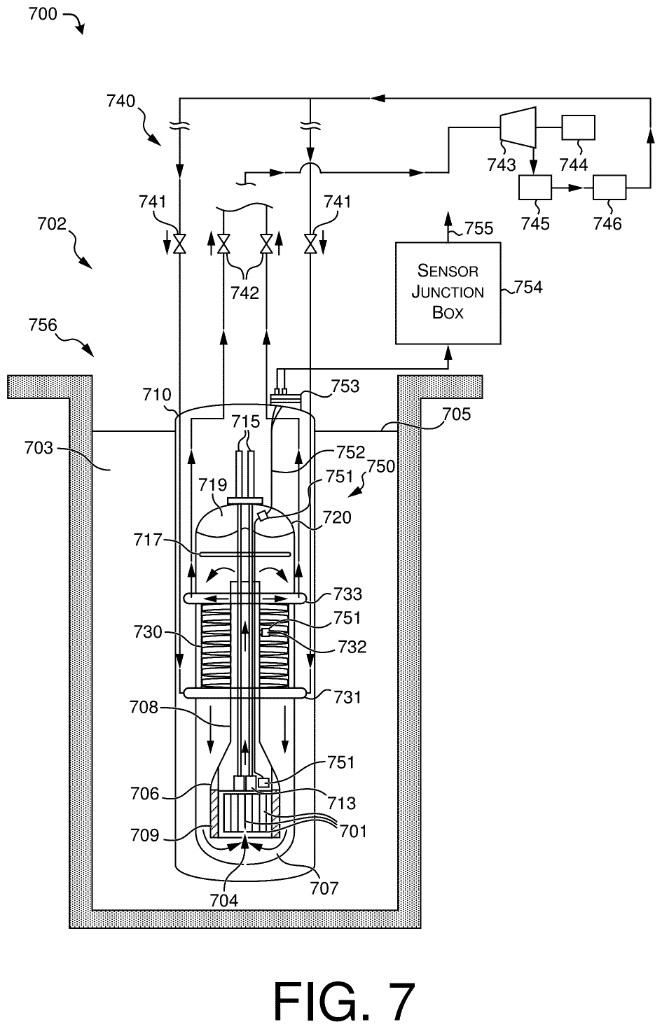

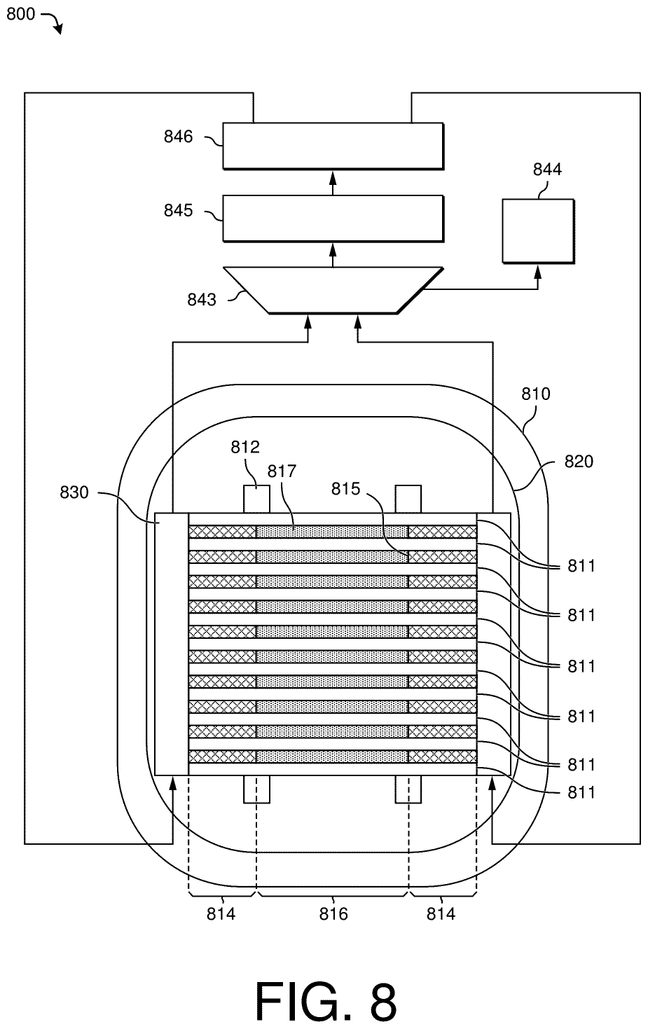

FIG. 1 schematically illustrates a representation of an integrated energy system (IES) 100 that includes a small modular nuclear reactor (SMR) system integrated with a Hydrothermal Decomposition process and Methanol (CH₃OH) production system. The IES 100 may include a Power Plant 102, a Hydrothermal Decomposition Plant 104, a Carbon Dioxide Conversion Plant 108, a Methanol Synthesis Reactor 112, and auxiliary equipment such as heaters, compressors, pumps, and control systems necessary to operate the IES 100 safely and at the desired conditions. In embodiments the power plant 102 may include the power plant system 650 of FIG. 6, in accordance with additional embodiments of the present technology. The power plant 102 may include a nuclear power module (NPM) including one or more light water nuclear reactor (LWR), one or more SMR, and any reactor 700 of FIG. 7, reactor system 800 of FIG. 8, and system of nuclear reactors 600 of FIG. 6. In embodiments, the Power Plant 102 can be configured to provide electrical and/or thermal energy to the Hydrothermal Decomposition Plant 104, the Carbon Dioxide Conversion Plant 108, and the Methanol Synthesis Reactor 112.

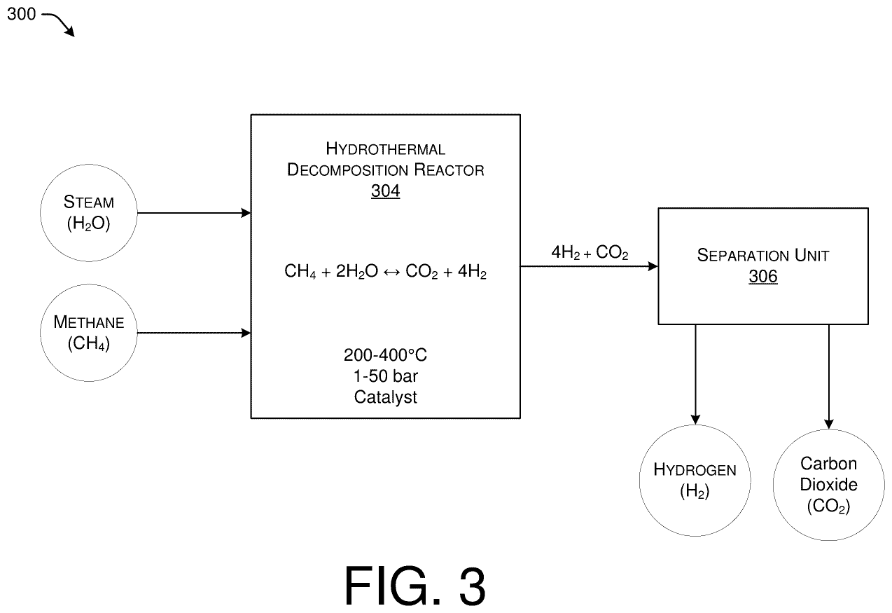

In the illustrated embodiment, the Power Plant 102 may be configured to provide steam, thermal energy, and/or electricity to the Hydrothermal Decomposition Plant 104. In embodiments, the Hydrothermal Decomposition Plant 104 may include the Hydrothermal Decomposition Plant 300 described in FIG. 3. In embodiments, the Hydrothermal Decomposition Plant 104 can be configured to receive Methane (CH₄) and steam (H₂O), and to produce Hydrogen (H₂) and Carbon dioxide (CO₂). In embodiments, the Methane (CH₄) may be received from a supplier, or maybe generated onsite. For example, the Methane (CH₄) may be obtained from purifying natural gas or other economical sources. In embodiments, the steam (H₂O) may include steam supplied directly from the Power Plant 102. In embodiments, the steam (H₂O) may include steam from a clean water source other than the Power Plant 102, for example clean water from a water treatment plant that has been heated and/or treated to the meet the desired operating conditions. In embodiments, the steam (H₂O) may be heated to the desired operating temperature using auxiliary heaters, such as electric heaters and/or heat exchangers. In embodiments, the steam (H₂O) may be compressed to the desired process pressure using auxiliary compressors or pumps. In embodiments, any auxiliary equipment within the IES 100, such as heaters, compressors, pumps, building lights, climate control, control systems, etc., can receive power from the Power Plant 102 and/or a power grid.

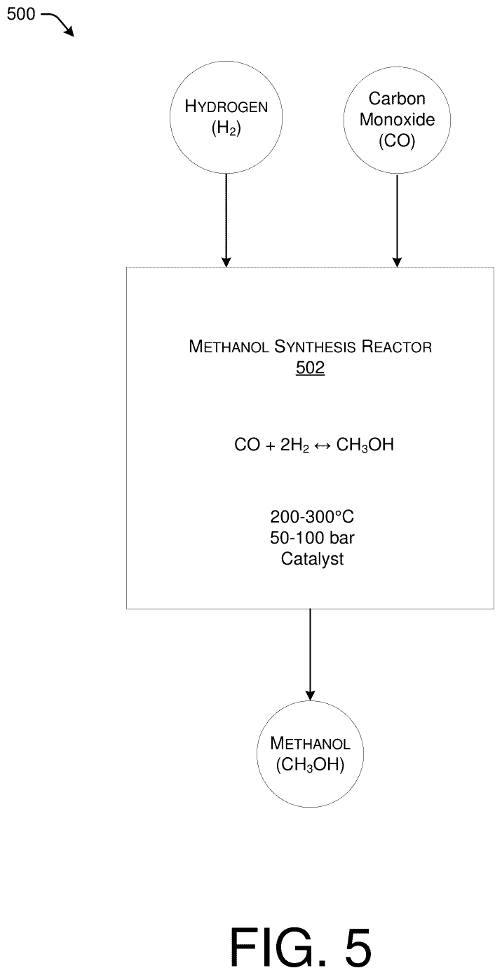

In embodiments, the Hydrogen (H₂) produced in the Hydrothermal Decomposition Plant 104 may be recycled within the IES 100, used in downstream processes connected to the IES 100, removed from the IES 100, stored for future use, and/or sold. For example, a first portion of the Hydrogen (H₂) can be removed from the IES 100 while a second portion of the Hydrogen (H₂) is fed to a downstream process, such as the Methanol Synthesis Reactor 112. In embodiments, the Methanol Synthesis Reactor 112 may include the Methanol Synthesis Reactor 502 described in FIG. 5.

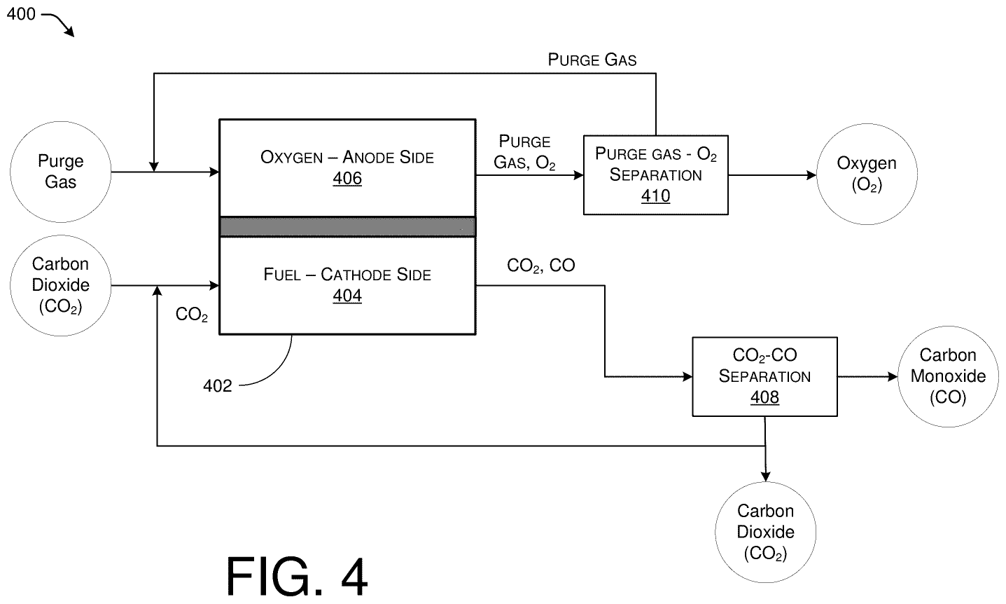

In embodiments, the Carbon Dioxide (CO₂) produced in the Hydrothermal Decomposition Plant 104 may be recycled within the IES 100, used in downstream processes connected to the IES 100, removed from the IES 100, stored for future use, and/or sold. For example, the Carbon Dioxide (CO₂) can be fed to the Carbon Dioxide Conversion Plant 108. In embodiments, the Carbon Dioxide Conversion Plant 108 may include the Carbon Dioxide (CO₂) Conversion Plant 400 described in FIG. 4. In embodiments, the Carbon Dioxide Conversion Plant 108 is configured to produce Carbon Monoxide (CO), Oxygen (O₂) and Carbon Dioxide (CO₂) from process inputs within the IES 100, for example, electricity from the Power Plant 102 and Carbon Dioxide (CO₂) from the Hydrothermal Decomposition Pant 104. In embodiments, the Carbon Dioxide (CO₂) produced in the Carbon Dioxide Conversion Plant 108 is configured to be recycled back to the input of the Carbon Dioxide Conversion Plant 108. In embodiments, the Carbon Dioxide Conversion Plant 108 is configured to produce Carbon Monoxide (CO) and Oxygen (O₂), and Carbon Dioxide (CO₂) from sources outside of the IES 100, such as purge gas and/or Carbon Dioxide (CO₂), for example purified Carbon Dioxide (CO₂) from an emission source.

In embodiments, the Methanol Synthesis Reactor 112 is configured to produce Methanol (CH₃OH) via Carbon Monoxide (CO) hydrogenation using inputs from within the IES 100, for example, electricity from the Power Plant 102, Hydrogen (H₂) from the Hydrothermal Decomposition Pant 104, and Carbon Monoxide (CO) from the Carbon Dioxide Conversion Plant 108. In embodiments, the Methanol produced within the Methanol Synthesis Reactor 112 may be, used in downstream processes connected to the IES 100, removed from the IES 100, stored for future use, and/or sold. In embodiments, intermediate streams, and products within the IES 100 may be recycled in order to optimize overall yield and efficiency. In embodiments resources from outside the IES 100, such as from storage and/or from a supplier, may be added to any one or more process within the IES 100 in quantities sufficient to optimize overall yield and efficiency and to minimize byproduct and/or waste production.

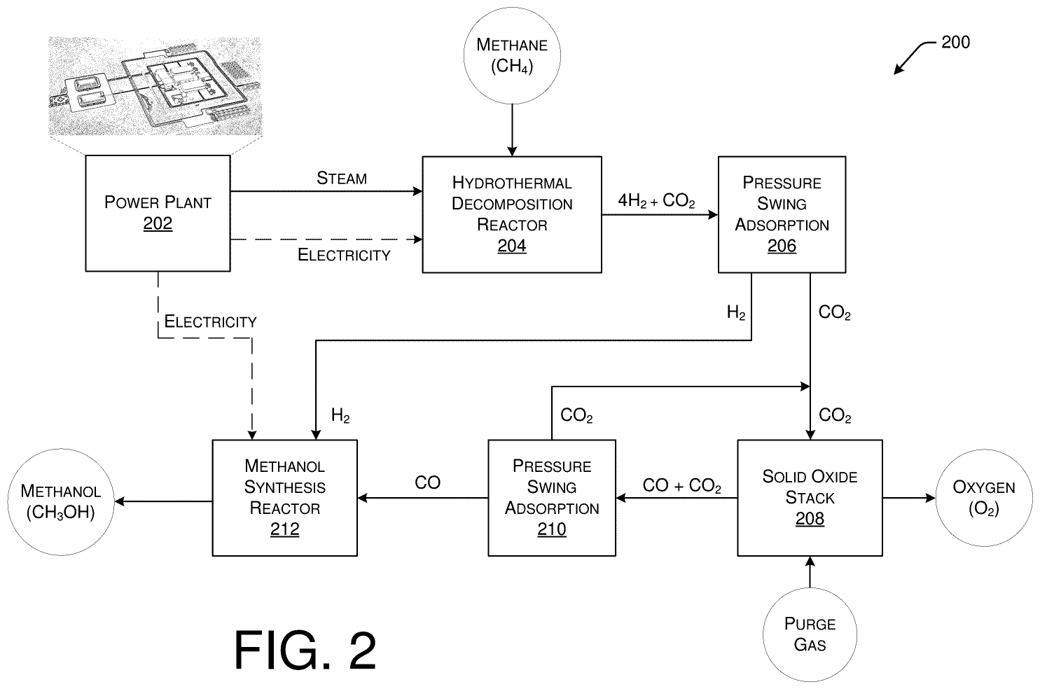

FIG. 2 schematically illustrates a representation of an integrated energy system (IES) 200 that includes a small modular nuclear reactor (SMR) system integrated with a Hydrothermal Decomposition process and Methanol (CH₃OH) production system. The IES 200 may include a Power Plant 202, a Hydrothermal Decomposition Reactor 204, a Pressure Swing Adsorption (PSA) unit 206, a Solid Oxide Stack 208, a Pressure Swing Adsorption (PSA) unit 210, a Methanol Synthesis Reactor 212, and auxiliary equipment such as heaters, compressors, pumps, and control systems necessary to operate the IES 200 safely and at the desired conditions. In embodiments the power plant 202 may include the power plant system 650 of FIG. 6, in accordance with additional embodiments of the present technology. The power plant 202 may include a nuclear power module (NPM) including one or more light water nuclear reactor (LWR), one or more SMR, and any reactor 700 of FIG. 7, reactor system 800 of FIG. 8, and system of nuclear reactors 600 of FIG. 6. In embodiments, the Power Plant 202 can be configured to provide electrical, thermal energy and steam to the Hydrothermal Decomposition Reactor 204, the Pressure Swing Adsorption (PSA) unit 206, the Solid Oxide Stack 208, the Pressure Swing Adsorption (PSA) unit 210, and the Methanol Synthesis Reactor 212.

In the illustrated embodiment, the Power Plant 202 may be configured to provide steam, thermal energy, and electricity to the Hydrothermal Decomposition Reactor 204. In embodiments, the Hydrothermal Decomposition Reactor 204 may include the Hydrothermal Decomposition Reactor 304 described in FIG. 3. In embodiments, the Hydrothermal Decomposition Reactor 204 can be configured to receive Methane (CH₄) and steam (H₂O), and to produce Hydrogen (H₂) and Carbon dioxide (CO₂). In embodiments, the Methane (CH₄) may be received from a supplier, or maybe generated onsite. For example, the Methane (CH₄) may be obtained from purifying natural gas or other economical sources. In embodiments, the steam (H₂O) may include steam supplied directly from the Power Plant 202. In embodiments, the steam (H₂O) may include steam from a clean water source other than the Power Plant 202, for example clean water from a water treatment plant that has been heated and/or treated to the meet the desired operating conditions. In embodiments, the steam (H₂O) may be heated to the desired operating temperature using auxiliary heaters, such as electric heaters and/or heat exchangers. In embodiments, the steam (H₂O) may be compressed to the desired process pressure using auxiliary compressors or pumps. In embodiments, any auxiliary equipment within the IES 200, such as heaters, compressors, pumps, building lights, climate control, control systems, etc., can receive power from the Power Plant 202 and/or a power grid.

In embodiments, the Hydrogen (H₂) and Carbon Dioxide (CO₂) produced in the Hydrothermal Decomposition Reactor 204 are output from the reactor in a single gas outlet stream. The gas outlet stream from the Hydrothermal Decomposition Reactor 204 can be fed to a separation unit, such as the Pressure Swing Adsorption unit 206, to separate the Hydrogen (H₂) and Carbon Dioxide (CO₂) into two purified gas streams, for example a purified Hydrogen (H₂) stream and a purified Carbon Dioxide (CO₂) stream. In embodiments, the purified Hydrogen (H₂) stream and/or the purified Carbon Dioxide (CO₂) stream may be recycled within the IES 200, used in downstream processes connected to the IES 200, removed from the IES 200, stored for future use, and/or sold. For example, a first portion of the Hydrogen (H₂) can be removed from the IES 200 while a second portion of the purified Hydrogen (H₂) is fed to a downstream process, such as the Methanol Synthesis Reactor 212. In embodiments, the Methanol Synthesis Reactor 212 may include the Methanol Synthesis Reactor 502 described in FIG. 5.

In embodiments, the purified Carbon Dioxide (CO₂) can be fed to the Solid Oxide Stack 208. In embodiments, the Solid Oxide Stack 208 may include the Solid Oxide Stack 402 described in FIG. 4. In embodiments, the Solid Oxide Stack 208 is configured to produce Carbon Dioxide (CO₂), Carbon Monoxide (CO) and Oxygen (O₂) from process inputs within the IES 200, for example, electricity from the Power Plant 202 and Carbon Dioxide (CO₂) from the Pressure Swing Adsorption unit 206. In embodiments, the Solid Oxide Stack 208 is configured to produce Carbon Dioxide (CO₂), Carbon Monoxide (CO) and Oxygen (O₂) from sources outside of the IES 200, such as purge gas and/or Carbon Dioxide (CO₂), for example purified Carbon Dioxide (CO₂) from an emission source.

In embodiments, the Carbon Monoxide (CO) produced in the Solid Oxide Stack 208 leaves the stack mixed with unconverted Carbon Dioxide (CO₂) in a single gas outlet stream. The mixed gas outlet stream from the Solid Oxide Stack 208 can be fed to a separation unit, such as the Pressure Swing Adsorption unit 210, to separate the Carbon Monoxide (CO) and Carbon Dioxide (CO₂) into two purified gas streams, for example a purified Carbon Monoxide (CO) stream and a purified Carbon Dioxide (CO₂) stream. In embodiments, the purified Carbon Monoxide (CO) stream and/or the purified Carbon Dioxide (CO₂) stream can be recycled within the IES 200, used in downstream processes connected to the IES 200, removed from the IES 200, stored for future use, and/or sold. In embodiments, the purified Carbon Monoxide (CO) stream can be fed to the Methanol Synthesis Reactor 212. In embodiments, the purified Carbon Dioxide (CO₂) stream can be recycled to the input of the Solid Oxide Stack 208.

In embodiments, the Methanol Synthesis Reactor 212 is configured to produce Methanol (CH₃OH) via Carbon Monoxide (CO) hydrogenation using inputs from within the IES 200, for example, electricity from the Power Plant 202, Hydrogen (H₂) from the Pressure Swing Adsorption unit 206, and Carbon Monoxide (CO) from the Pressure Swing Adsorption unit 210. In embodiments, the Methanol produced within the Methanol Synthesis Reactor 212 may be, used in downstream processes connected to the IES 200, removed from the IES 200, stored for future use, and/or sold.

FIG. 3 is a schematic diagram of a Hydrothermal Decomposition Plant 300. In the illustrated embodiment, the Hydrothermal Decomposition Plant 300 includes one or more Hydrothermal Decomposition Reactor(s) 304, one or more Separation Unit(s) 306, and auxiliary equipment such as heaters, compressors, pumps, and control systems necessary to operate the Hydrothermal Decomposition Plant 300 safely and at the desired conditions. In embodiments, the Hydrothermal Decomposition Plant 300 may be configured to receive steam, thermal energy, and/or electricity from a power plant, such as a nuclear power module (NPM) including one or more light water nuclear reactor (LWR), one or more small modular nuclear reactor (SMR), and any reactor 700 of FIG. 7, reactor system 800 of FIG. 8, and system of nuclear reactors 600 of FIG. 6.

In embodiments, the Hydrothermal Decomposition Reactor 304 can be configured to receive Methane (CH₄) and steam (H₂O), and to produce Hydrogen (H₂) and Carbon Dioxide (CO₂). In embodiments, the Methane (CH₄) may be received from a supplier, or maybe generated onsite. For example, the Methane (CH₄) may be obtained from purifying natural gas or other economical sources. In embodiments, the steam (H₂O) can include steam supplied directly from a nuclear power plant, in accordance with embodiments of the present disclosure. In embodiments, the steam (H₂O) may include steam from a clean water source, for example clean water from a water treatment plant that has been heated and/or treated to the meet the desired operating conditions. In embodiments, the steam (H₂O) may be heated to the desired operating temperature using auxiliary heaters, such as electric heaters and/or heat exchangers. In embodiments, the steam (H₂O) may be compressed to the desired process pressure using auxiliary compressors or pumps. In embodiments, any auxiliary equipment within the Hydrothermal Decomposition Plant 300, such as heaters, compressors, pumps, building lights, climate control, control systems, etc., can receive power from a nuclear power plant in accordance with embodiments of the present disclosure, and/or a power grid.

In embodiments, Methane (CH₄) received within the Hydrothermal Decomposition Plant 300 can be reacted with steam (H₂O), for example in Hydrothermal Decomposition Reactor 304, to produce Hydrogen (H₂) and Carbon Dioxide (CO₂) according to Equation 3:

$${CH}_{4} + 2 {H}_{2} O ↔ {CO}_{2} + 4 {H}_{2} \qquad ( 3 )$$

The hydrothermal decomposition of Methane (CH₄) of the present disclosure is a process in which the operating temperature is in the range of about 200-400° C., the pressure is about 1-50 bar, and the reaction occurs in the presence of a catalyst, for example a group 8 to 10 metal-based catalyst such as a nickel (Ni) based catalyst and/or a Ruthenium (Ru) based catalyst. In embodiments, the feed composition to the Hydrothermal Decomposition Reactor 304 may include 10-100% Methane (CH₄) and 10-100% Steam (H₂O). In embodiments, the steam (H₂O) and the Methane (CH₄) may be fed to the Hydrothermal Decomposition Reactor 304 at a ratio of 1:4. In embodiments, thermal energy and/or power from a nuclear power plant in accordance with embodiments of the present disclosure can be directed to the Hydrothermal Decomposition Reactor 304 as needed to maintain the desired operating conditions.

In embodiments, the Hydrogen (H₂) and Carbon Dioxide (CO₂) produced in the Hydrothermal Decomposition Reactor 304 are output from the reactor in a single gas outlet stream. The gas outlet stream from the Hydrothermal Decomposition Reactor 304 can be fed to a Separation Unit 306 to separate the Hydrogen (H₂) and Carbon Dioxide (CO₂) into two purified gas streams, for example a purified Hydrogen (H₂) stream and a purified Carbon Dioxide (CO₂) stream. In embodiments, the Separation Unit 306 may include a Pressure Swing Adsorption (PSA) unit, or any separation unit suitable for separating a mixed Hydrogen (H₂) and Carbon Dioxide (CO₂) gas stream into two purified gas streams. In embodiments, the purified Hydrogen (H₂) stream and/or the purified Carbon Dioxide (CO₂) stream may be used in downstream processes connected to the Hydrothermal Decomposition Plant 300, removed from the Hydrothermal Decomposition Plant 300, stored for future use, and/or sold. In embodiments, a first portion of the Hydrogen (H₂) can be removed from the Hydrothermal Decomposition Plant 300, and a second portion of the purified Hydrogen (H₂) is fed to a downstream process, such as a Methanol (CH₃OH) synthesis reactor. In embodiments, the purified Carbon Dioxide (CO₂) stream can be fed to a downstream process such as a solid oxide stack.

FIG. 4 is a block diagram of a Carbon Dioxide (CO₂) Conversion Plant 400, in accordance with embodiments of the present disclosure. In embodiments, the Carbon Dioxide (CO₂) Conversion Plant 400 may include one or more Solid Oxide Stack 402, a CO₂—CO Separation process 408, a Purge Gas-O₂ Separation process 410, and auxiliary equipment such as heaters, compressors, pumps, and control systems necessary to operate the Carbon Dioxide (CO₂) Conversion Plant 400 safely and at the desired conditions. In embodiments, the Hydrothermal Decomposition Plant 300 may be configured to receive steam, thermal energy, and/or electricity from a power plant, such as a nuclear power module (NPM) including one or more light water nuclear reactor (LWR), one or more small modular nuclear reactor (SMR), and any reactor 700 of FIG. 7, reactor system 800 of FIG. 8, and system of nuclear reactors 600 of FIG. 6.

A Solid Oxide Stack 402 of the present invention may be configured to perform the electrochemical Carbon Dioxide (CO₂) reduction for Carbon Monoxide (CO) and Oxygen (O₂), as described, for example, in Kungas, J. Electrochem. Soc. 167, 2020 and WO2014/154253. In embodiments, the Solid Oxide Stack 402 may include a solid ceramic material, for example stabilized zirconias, such as yttria-stabilized zirconia (YSZ, a solid solution of Y₂O₃ and ZrO₂) and scandia-stabilized zirconia (ScSZ), as well as doped cerias, such as gadolinia-doped ceria (abbreviated either as GDC) or samaria-doped ceria (SDC). The Solid Oxide Stack 402 may include one or more of a Solid Oxide Electrolysis Cell (SOEC) or a Co-Electrolysis cell configured to produce Carbon Monoxide (CO) and Oxygen (O₂).

In embodiments, Carbon Dioxide (CO₂) is fed to the Solid Oxide Stack 402. In embodiments, the Carbon Dioxide (CO₂) may be fed to the Solid Oxide Stack 402 from storage, from a supplier, and/or directly from an upstream process. In embodiments, the Carbon Dioxide (CO₂) is purified Carbon Dioxide (CO₂) from a Hydrothermal Decomposition of Methane (HTDM) process, such as the Hydrothermal Decomposition Plant 400, and/or purified Carbon Dioxide (CO₂) from an emission source.

In embodiments, Carbon Dioxide (CO₂) is fed to the fuel side 404, i.e., the cathode side, of the Solid Oxide Stack 402 with an applied current and Oxygen (O₂) is generated. The equation that governs the Carbon Dioxide (CO₂) reduction for Carbon Monoxide (CO) and Oxygen (O₂) production is shown in Equation 6:

$$2 {CO}_{2} \to {CO}_{2} + C O + 1 / 2 {O}_{2} \qquad ( 6 )$$

Note the molar ratio of Carbon Dioxide (CO₂) gas and Carbon Monoxide (CO) gas in Equation 6 is 1:1. Oxygen (O₂) generated in the reaction is then transported to the oxygen side 406, i.e., the anode side, of the Solid Oxide Stack 402. In embodiments, a purge gas, such as Carbon Dioxide (CO₂), air, or nitrogen may be used to flush the oxygen side 406 of the Solid Oxide Stack 402. In embodiments, Carbon Dioxide (CO₂) can be used to flush the oxygen side 406, instead of air, to mitigate the leakage of undesired gases, such as Nitrogen (N₂), into the fuel side 404 of the Solid Oxide Stack 402. Flushing the oxygen side 406 of the Solid Oxide Stack 402 with Carbon Dioxide (CO₂) gas has two advantages, more specifically (1) enhancing the oxygen production concentration and (2) providing means for feeding energy into the Solid Oxide Stack 402.

In embodiments, the Solid Oxide Stack 402 is operated at elevated temperatures (e.g., ˜600° C.). Inlet gas to the fuel side 404 and/or flush gas to the oxygen side 406 may be heated, for example in one or more auxiliary heaters, prior to entering the Solid Oxide Stack 402. In embodiments, Joule heat, i.e., the heat produced when current is passed through the Solid Oxide Stack 402, may supply some or all of the heat necessary for the Solid Oxide Stack 402. In embodiments, Joule heat, auxiliary heaters, and/or means of heating known in the art may be used in combination to provide optimum operating conditions for the Solid Oxide Stack 402. In embodiments, the Carbon Dioxide (CO₂) Conversion Plant 400 may receive power and/or thermal energy from a nuclear power plant in accordance with embodiments of the present disclosure.

In embodiments, the product stream from the fuel side 404 of the Solid Oxide Stack 402, for example a gas stream containing Carbon Monoxide (CO) mixed with Carbon Dioxide (CO₂), may then be passed through a CO₂—CO Separation process 408 to separate the Carbon Monoxide (CO) and the Carbon Dioxide (CO₂). In embodiments, the CO₂—CO Separation process 408 may include one or more separation units, such as pressure swing adsorption (PSA), temperature swing adsorption (TSA), membrane separation, and cryogenic separation technology. Carbon Dioxide (CO₂) from the CO₂—CO Separation process 408 may be recycled within the Carbon Dioxide (CO₂) Conversion Plant 400, for example to the inlet of the Solid Oxide Stack 402, and/or used in downstream processes, such as in a Methanol (CH₃OH) production process. In embodiments, Carbon Monoxide (CO) from the CO₂—CO Separation process 408 may be recycled within the Carbon Dioxide (CO₂) Conversion Plant 400 and/or used in downstream processes, such as in a Methanol (CH₃OH) production process.

In embodiments, the product stream from the oxygen side 406 of the Solid Oxide Stack 402, for example a gas stream containing Oxygen (O₂) mixed with purge gas, may then be passed through a Purge Gas-O₂ Separation process 410 to separate the Oxygen (O₂) and the purge gas. In embodiments, the Purge Gas-O₂ Separation process 410 may include one or more separation units, such as PSA, adsorption, membrane separation, or any separation process sufficient to separate Oxygen (O₂) from the Purge Gas. In embodiments, the Purge gas from the Purge Gas-O₂ Separation process 410 may be recycled within the Carbon Dioxide (CO₂) Conversion Plant 400, for example to the inlet of the Solid Oxide Stack 402. Oxygen (O₂) from the Purge Gas-O₂ Separation process 410 may be used in downstream processes, further purified, stored, and/or sold, for example to hospitals and industry.

FIG. 5 is a schematic diagram of a Methanol Production Plant 500. In the illustrated embodiment, the Methanol Production Plant 500 includes one or more Methanol Synthesis Reactor(s) 502 and auxiliary equipment such as heaters, compressors, pumps, and control systems necessary to operate the Methanol Production Plant 500 safely and at the desired conditions. In embodiments, the Methanol Production Plant 500 may be configured to receive steam, thermal energy, and/or electricity from a power plant, such as a nuclear power module (NPM) including one or more light water nuclear reactor (LWR), one or more small modular nuclear reactor (SMR), and any reactor 700 of FIG. 7, reactor system 800 of FIG. 8, and system of nuclear reactors 600 of FIG. 6.

In embodiments, the Methanol Synthesis Reactor 502 can be configured to receive Hydrogen (H₂) and Carbon Monoxide (CO), and to produce Methanol (CH₃OH). In embodiments, the Methanol Synthesis Reactor 502 is configured to produce Methanol (CH₃OH) via Carbon Monoxide (CO) hydrogenation over a catalyst, such as a Nickle Oxide (NiO₂) catalyst, and/or a Copper (Cu) and zinc oxide (ZnO) based catalyst (Cu/ZnO), at a temperature between 20° and 300° C., for example about 250° C., and at a pressure between 5-10 MPa (50-100 bar). The reaction is shown in Equation 5:

$$CO + 2 {H}_{2} {↔}^{Cu / ZnO} {CH}_{3} OH \qquad ( 5 )$$

In embodiments, the Methanol Synthesis Reactor 502 receives purified Hydrogen (H₂) from a source such as the Hydrothermal Decomposition Plant 300, and purified Carbon Monoxide (CO) from a source such as the Carbon Dioxide (CO₂) Conversion Plant 400. In embodiments, the Methanol Synthesis Reactor 502 can be configured to receive purified Hydrogen (H₂) and/or Carbon Monoxide (CO) from a supplier.

There is significant advantage to the production of Methanol (CH₃OH) via Carbon Monoxide (CO) hydrogenation as described herein. By introducing purified Hydrogen (H₂) and purified Carbon Monoxide (CO) to the Methanol Synthesis Reactor 502 there is no water present in the process, and the hydrogenation process can take place at low-temperature, and therefore can be sustained and continuous without interruption and additional cost to change out deactivated catalyst. This advantage and the improved efficiency of this process described herein contributes to the production of “green” Methanol (CH₃OH).

In embodiments, the Methanol (CH₃OH) produced within the Methanol Synthesis Reactor 502 may be used in downstream processes connected to the Methanol Synthesis Plant 500, removed from the Methanol Synthesis Plant 500, stored for future use, and/or sold.

FIG. 6 is FIG. 6 is a schematic view of a nuclear power plant system 650 (“power plant system 650”) including multiple nuclear reactors 600 (individually identified as first through twelfth nuclear reactors 600A-L, respectively) in accordance with embodiments of the present technology. The power plant system 650 can be a permanent or temporary installation built at or near (e.g., roughly 1 km from) the location of an industrial process facility or can be a mobile or partially mobile system that is moved to and assembled at or near the location of the industrial process facility. More generally, the power plant can be local (e.g., positioned at or near) to the industrial processes/operations it supports. For example, the power plant can be located within 0.4 km (0.25 mile), within 0.8 km (0.5 mile), within 3.22 km (2 miles), within 4.82 km (3 miles), or within 8.1 km (5 miles) of the industrial processes/operations it supports. In embodiments, the power plant system 650 is configured to supply a portion of electricity to a power grid.

In embodiments, the power plant system 650 may produce and deliver electrical power to the power grid during peak times or anytime that there is a demand for energy production. For example, when consumer energy demand imposes a high energy demand on the power grid (“peak times”), the power plant system 650 may be configured to produce and provide the energy necessary for the power grid to meet the high consumer demand during peak times. In embodiments, the power plant system 650 may be configured to provide energy directly to the power grid as required to meet energy demand due to factors other than increased energy demand during peak times (e.g., an energy producing plant that provides energy to the power grid may be offline and unable to provide energy, which creates an increased energy production demand without an increased demand for consumer electrical power).

Each of the nuclear reactors 600 can be similar to, or identical to, the nuclear reactor system 700 and/or the nuclear reactor system 800 described in detail below with reference to FIG. 7 and FIG. 8. The power plant system 650 can be “modular” in that each of the nuclear reactors 600 can be operated separately to provide an output, such as electricity or steam. In embodiments, the power plant system may include Small Modular Reactors (SMRs), a microreactor, or other types of advanced reactors. In one embodiment, the nuclear reactor module may be a light pressurized water reactor (PWR) but may be different types of reactors utilizing a variety of fuels and located on terrestrial, maritime, and extraterrestrial sites. The power plant system 650 can include fewer than twelve of the nuclear reactors 600 (e.g., two, three, four, five, six, seven, eight, nine, ten, or eleven of the nuclear reactors 600), or more than twelve of the nuclear reactors 600. The power plant system 650 can be a permanent installation or can be mobile (e.g., mounted on a truck, tractor, mobile platform, and/or the like). In the illustrated embodiment, each of the nuclear reactors 600 can be positioned within a common housing 651, such as a reactor plant building, and controlled and/or monitored via a control room 652.

Each of the nuclear reactors 600 can be coupled to a corresponding electrical power conversion system 640 (individually identified as first through twelfth electrical power conversion systems 640A-L, respectively). The electrical power conversion systems 640 can include one or more devices that generate electrical power or some other form of usable power from steam generated by the nuclear reactors 600. For example, the electrical power conversion systems 640 can include features that are similar or identical to the power conversion system 740 described in detail below with reference to FIG. 7. In some embodiments, multiple ones of the nuclear reactors 600 can be coupled to the same one of the electrical power conversion systems 640 and/or one or more of the nuclear reactors 600 can be coupled to multiple ones of the electrical power conversion systems 640 such that there is not a one-to-one correspondence between the nuclear reactors 600 and the electrical power conversion systems 640.

The electrical power conversion systems 640 can be further coupled to an electrical power transmission system 654 via, for example, an electrical power bus 653. The electrical power transmission system 654 and/or the electrical power bus 653 can include one or more transmission lines, transformers, and/or the like for regulating the current, voltage, and/or other characteristic(s) of the electricity generated by the electrical power conversion systems 640. The electrical power transmission system 654 can route electricity via a plurality of electrical output paths 655 (individually identified as electrical output paths 655A-N) to one or more end users and/or end uses, such as different electrical loads of an integrated energy system as described in greater detail herein.

The power plant system 650 can be configured in a first operating state to provide electricity to a water treatment plant (e.g., via one or more of the electrical output paths 655 from the electrical power transmission system 654). The water treatment plant can route the produced high-quality water to the power plant system 650, and the power plant system 650 can use the water to produce high-quality steam. For example, the produced water can be used as a secondary coolant in a steam generator of one or more of the nuclear reactors 600. In some embodiments, the water treatment plant can be omitted and the power plant system 650 can utilize water from other sources to generate steam.

Each of the nuclear reactors 600 can further be coupled to a steam transmission system 656 via, for example, a steam bus 657. The steam bus 657 can route steam generated from the nuclear reactors 600 to the steam transmission system 656 which in turn can route the steam via a plurality of steam output paths 658 (individually identified as steam output paths 658A-N) to one or more end users and/or end uses, such as different steam inputs of an integrated energy system as described in greater detail below.

In some embodiments, the nuclear reactors 600 can be individually controlled (e.g., via the control room 652) to provide steam to the steam transmission system 656 and/or steam to the corresponding one of the electrical power conversion systems 640 to provide electricity to the electrical power transmission system 654. In some embodiments, the nuclear reactors 600 are configured to provide steam either to the steam bus 657 or to the corresponding one of the electrical power conversion systems 640 and can be rapidly and efficiently switched between providing steam to either. Accordingly, in some aspects of the present technology the nuclear reactors 600 can be modularly and flexibly controlled such that the power plant system 650 can provide differing levels/amounts of electricity via the electrical power transmission system 654 and/or steam via the steam transmission system 656. For example, where the power plant system 650 is used to provide electricity and steam to one or more industrial process-such as various components of the integrated energy systems described in the detail below—the nuclear reactors 600 can be controlled to meet the differing electricity and steam requirements of the industrial processes.

As one example, during a first operational state of an integrated energy system employing the power plant system 650, a first subset of the nuclear reactors 600 (e.g., the first through sixth nuclear reactors 600A-F) can be configured to provide steam to the steam transmission system 656 for use in the first operational state of the integrated energy system, while a second subset of the nuclear reactors 600 (e.g., the seventh through twelfth nuclear reactors 600G-L) can be configured to provide steam to the corresponding ones of the electrical power conversion systems 640 (e.g., the seventh through twelfth electrical power conversion systems 640G-L) to generate electricity for the first operational state of the integrated energy system. Then, during a second operational state of the integrated energy system when a different (e.g., greater or lesser) amount of steam and/or electricity is required, some or all the first subset of the nuclear reactors 600 can be switched to provide steam to the corresponding ones of the electrical power conversion systems 640 (e.g., the seventh through twelfth electrical power conversion systems 640G-L) and/or some or all of the second subset of the nuclear reactors 600 can be switched to provide steam to the steam transmission system 656 to vary the amount of steam and electricity produced to match the requirements/demands of the second operational state. Other variations of steam and electricity generation are possible based on the needs of the integrated energy system. That is, the nuclear reactors 600 can be dynamically/flexibly controlled during other operational states of an integrated energy system to meet the steam and electricity requirements of the operational state.

In contrast, some conventional nuclear power plant systems can typically generate a fixed amount of either steam or electricity for output, and cannot be modularly controlled to provide varying levels of steam and electricity for output. Moreover, it is typically difficult (e.g., expensive, time consuming, etc.) to switch between steam generation and electricity generation in conventional nuclear power plant systems. Specifically, for example, it is typically extremely time consuming to switch between steam generation and electricity generation in prototypical large nuclear power plant systems.

The nuclear reactors 600 can be individually controlled via one or more operators and/or via a computer system. Accordingly, many embodiments of the technology described herein may take the form of computer- or machine- or controller-executable instructions, including routines executed by a programmable computer or controller. Those skilled in the relevant art will appreciate that the technology can be practiced on computer/controller systems other than those shown and described herein. The technology can be embodied in a special-purpose computer, controller or data processor that is specifically programmed, configured or constructed to perform one or more of the computer-executable instructions described below. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, micro-computers and the like). Information handled by these computers can be presented at any suitable display medium, including a liquid crystal display (LCD).

The technology can also be practiced in distributed environments, where tasks or modules are performed by remote processing devices that are linked through a communications network. In a distributed computing environment, program modules or subroutines may be located in local and remote memory storage devices. Aspects of the technology described herein may be stored or distributed on computer-readable media, including magnetic or optically readable or removable computer disks, as well as distributed electronically over networks. Data structures and transmissions of data particular to aspects of the technology are also encompassed within the scope of the embodiments of the technology.

FIG. 7 and FIG. 8 illustrate representative nuclear reactors that may be included in embodiments of the present technology. The nuclear reactor module of FIG. 7 or FIG. 8 may be a small modular reactor (SMR), a microreactor, or other types of advanced reactors. In one embodiment, the nuclear reactor module may be a light pressurized water reactor (PWR) but may be different types of reactors utilizing a variety of fuels and located on terrestrial, maritime, and extraterrestrial sites. FIG. 7 is a partially schematic, partially cross-sectional view of a nuclear reactor system 700 configured in accordance with embodiments of the present technology. The system 700 can include a power module 702 having a reactor core 704 in which a controlled nuclear reaction takes place. Accordingly, the reactor core 704 can include one or more fuel assemblies 701. The fuel assemblies 701 can include fissile and/or other suitable materials. Heat from the reaction generates steam at a steam generator 730, which directs the steam to a power conversion system 740. The power conversion system 740 generates electrical power, and/or provides other useful outputs, such as super-heated steam. A sensor system 750 is used to monitor the operation of the power module 702 and/or other system components. The data obtained from the sensor system 750 can be used in real time to control the power module 702, and/or can be used to update the design of the power module 702 and/or other system components.

The power module 702 includes a containment vessel 710 (e.g., a radiation shield vessel, or a radiation shield container) that houses/encloses a reactor vessel 720 (e.g., a reactor pressure vessel, or a reactor pressure container), which in turn houses the reactor core 704. The containment vessel 710 can be housed in a power module bay 756. The power module bay 756 can contain a cooling pool 703 filled with water and/or another suitable cooling liquid. The bulk of the power module 702 can be positioned below a surface 705 of the cooling pool 703. Accordingly, the cooling pool 703 can operate as a thermal sink, for example, in the event of a system malfunction.

A volume between the reactor vessel 720 and the containment vessel 710 can be partially or completely evacuated to reduce heat transfer from the reactor vessel 720 to the surrounding environment (e.g., to the cooling pool 703). However, in other embodiments the volume between the reactor vessel 720 and the containment vessel 710 can be at least partially filled with a gas and/or a liquid that increases heat transfer between the reactor vessel 720 and the containment vessel 710. For example, the volume between the reactor vessel 720 and the containment vessel 710 can be at least partially filled (e.g., flooded with the primary coolant 707) during an emergency operation.

Within the reactor vessel 720, a primary coolant 707 conveys heat from the reactor core 704 to the steam generator 730. For example, as illustrated by arrows located within the reactor vessel 720, the primary coolant 707 is heated at the reactor core 704 toward the bottom of the reactor vessel 720. The heated primary coolant 707 (e.g., water with or without additives) rises from the reactor core 704 through a core shroud 706 and to a riser tube 708. The hot, buoyant primary coolant 707 continues to rise through the riser tube 708, then exits the riser tube 708 and passes downwardly through the steam generator 730. The steam generator 730 includes a multitude of conduits 732 that are arranged circumferentially around the riser tube 708, for example, in a helical pattern, as is shown schematically in FIG. 7. The descending primary coolant 707 transfers heat to a secondary coolant (e.g., water) within the conduits 732, and descends to the bottom of the reactor vessel 720 where the cycle begins again. The cycle can be driven by the changes in the buoyancy of the primary coolant 707, thus reducing or eliminating the need for pumps to move the primary coolant 707.

The steam generator 730 can include a feedwater header 731 at which the incoming secondary coolant enters the steam generator conduits 732. The secondary coolant rises through the conduits 732, converts to vapor (e.g., steam), and is collected at a steam header 733. The steam exits the steam header 733 and is directed to the power conversion system 740.

The power conversion system 740 can include one or more steam valves 742 that regulate the passage of high pressure, high temperature steam from the steam generator 730 to a steam turbine 743. The steam turbine 743 converts the thermal energy of the steam to electricity via a generator 744. The low-pressure steam exiting the turbine 743 is condensed at a condenser 745, and then directed (e.g., via a pump 746) to one or more feedwater valves 741. The feedwater valves 741 control the rate at which the feedwater re-enters the steam generator 730 via the feedwater header 731. In other embodiments, the steam from the steam generator 730 can be routed for direct use in an industrial process, such as a hydrogen and oxygen production plant, a chemical production plant, and/or the like, as described in detail in this application. Accordingly, steam exiting the steam generator 730 can bypass the power conversion system 740.

The power module 702 includes multiple control systems and associated sensors. For example, the power module 702 can include a hollow cylindrical reflector 709 that directs neutrons back into the reactor core 704 to further the nuclear reaction taking place therein. Control rods 713 are used to modulate the nuclear reaction and are driven via fuel rod drivers 715. The pressure within the reactor vessel 720 can be controlled via a pressurizer plate 717 (which can also serve to direct the primary coolant 707 downwardly through the steam generator 730) by controlling the pressure in a pressurizing volume 719 positioned above the pressurizer plate 717.

The sensor system 750 can include one or more sensors 751 positioned at a variety of locations within the power module 702 and/or elsewhere, for example, to identify operating parameter values and/or changes in parameter values. The data collected by the sensor system 750 can then be used to control the operation of the system 700, and/or to generate design changes for the system 700. For sensors positioned within the containment vessel 710, a sensor link 752 directs data from the sensors to a flange 753 (at which the sensor link 752 exits the containment vessel 710) and directs data to a sensor junction box 754. From there, the sensor data can be routed to one or more controllers and/or other data systems via a data bus 755.

FIG. 8 is a partially schematic, partially cross-sectional view of a nuclear reactor system 800 (“system 800”) configured in accordance with additional embodiments of the present technology. In some embodiments, the system 800 can include some features that are at least generally similar in structure and function, or identical in structure and function, to the corresponding features of the system 700 described in detail above with reference to FIG. 7 and can operate in a generally similar or identical manner to the system 700.

In the illustrated embodiment, the system 800 includes a reactor vessel 820 and a containment vessel 810 surrounding/enclosing the reactor vessel 820. In some embodiments, the reactor vessel 820 and the containment vessel 810 can be roughly cylinder-shaped or capsule-shaped. The system 800 further includes a plurality of heat pipe layers 811 within the reactor vessel 820. In the illustrated embodiment, the heat pipe layers 811 are spaced apart from and stacked over one another. In some embodiments, the heat pipe layers 811 can be mounted/secured to a common frame 812, a portion of the reactor vessel 820 (e.g., a wall thereof), and/or other suitable structures within the reactor vessel 820. In other embodiments, the heat pipe layers 811 can be directly stacked on top of one another such that each of the heat pipe layers 811 supports and/or is supported by one or more of the other ones of the heat pipe layers 811.

In the illustrated embodiment, the system 800 further includes a shield or reflector region 814 at least partially surrounding a core region 816. The heat pipes layers 811 can be circular, rectilinear, polygonal, and/or can have other shapes, such that the core region 816 has a corresponding three-dimensional shape (e.g., cylindrical, spherical). In some embodiments, the core region 816 is separated from the reflector region 814 by a core barrier 815, such as a metal wall. The core region 816 can include one or more fuel sources, such as fissile material, for heating the heat pipes layers 811. The reflector region 814 can include one or more materials configured to contain/reflect products generated by burning the fuel in the core region 816 during operation of the system 800. For example, the reflector region 814 can include a liquid or solid material configured to reflect neutrons and/or other fission products radially inward toward the core region 816. In some embodiments, the reflector region 814 can entirely surround the core region 816. In other embodiments, the reflector region 814 may partially surround the core region 816. In some embodiments, the core region 816 can include a control material 817, such as a moderator and/or coolant. The control material 817 can at least partially surround the heat pipe layers 811 in the core region 816 and can transfer heat therebetween.

In the illustrated embodiment, the system 800 further includes at least one heat exchanger 830 (e.g., a steam generator) positioned around the heat pipe layers 811. The heat pipe layers 811 can extend from the core region 816 and at least partially into the reflector region 814 and are thermally coupled to the heat exchanger 830. In some embodiments, the heat exchanger 830 can be positioned outside of or partially within the reflector region 814. The heat pipe layers 811 provide a heat transfer path from the core region 816 to the heat exchanger 830. For example, the heat pipe layers 811 can each include an array of heat pipes that provide a heat transfer path from the core region 816 to the heat exchanger 830. When the system 800 operates, the fuel in the core region 816 can heat and vaporize a fluid within the heat pipes in the heat pipe layers 811, and the fluid can carry the heat to the heat exchanger 830. The heat pipes in the heat pipe layers 811 can then return the fluid toward the core region 816 via wicking, gravity, and/or other means to be heated and vaporized once again.

In some embodiments, the heat exchanger 830 can be similar to the steam generator 730 of FIG. 7 and, for example, can include one or more helically-coiled tubes that wrap around the heat pipe layers 811. The tubes of the heat exchanger 830 can include or carry a working fluid (e.g., a coolant such as water or another fluid) that carries the heat from the heat pipe layers 811 out of the reactor vessel 820 and the containment vessel 810 for use in generating electricity, steam, and/or the like. For example, in the illustrated embodiment the heat exchanger 830 is operably coupled to a turbine 843, a generator 844, a condenser 845, and a pump 846. As the working fluid within the heat exchanger 830 increases in temperature, the working fluid may begin to boil and vaporize. The working fluid (e.g., steam) may be used to drive the turbine 843 to convert the thermal potential energy of the working fluid into electrical energy via the generator 844. The condenser 845 can condense the working fluid after it passes through the turbine 843, and the pump 846 can direct the working fluid back to the heat exchanger 830 where it can begin another thermal cycle. In other embodiments, steam from the heat exchanger 830 can be routed for direct use in an industrial process, such as a resource production plant, described in detail above. Accordingly, steam exiting the heat exchanger 830 can bypass the turbine 843, the generator 844, the condenser 845, the pump 846, etc.

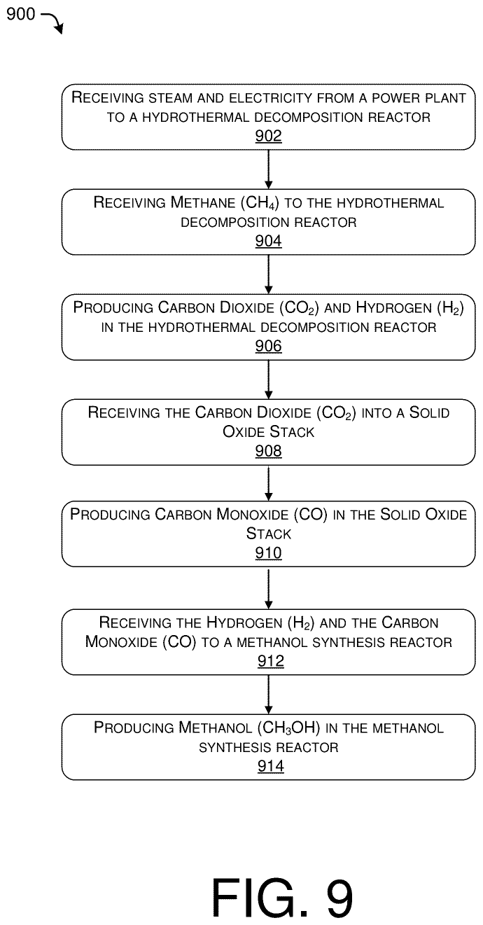

FIG. 9 illustrates and example process for producing Methanol 900. In various examples, the process can be performed by an Integrated Energy System (IES). The IES may include a power plant, such as the power plant 102 and the power plant system 650. The IES may also include a resource production plant, such as the Hydrothermal Decomposition Plant 104, the Electrolysis plant 108, and the Methanol synthesis reactor 112. In embodiments, the Hydrothermal Decomposition Plant 104, the Electrolysis plant 108, and the Methanol synthesis reactor 112 are configured to receive electricity from the power plant.

At 902, electricity and steam are received from a power plant, such as the power plant 102 and the power plant system 650, to a hydrothermal decomposition reactor. In embodiments, the power plant produces electricity and steam from nuclear energy. For example, the power plant may include one or more small modular nuclear reactor (SMRs) as described elsewhere in this disclosure. In embodiments, the power plant may be local to the resource production plant. For example, the power plant may be located within 0.4 km (0.25 mile), within 0.8 km (0.5 mile), within 3.22 km (2 miles), within 4.82 km (3 miles), or within 8.1 km (5 miles) of the resource production plant it supports. In embodiments, the power plant is configured to supply a portion of electricity to a power grid.

At 904, Methane (CH₄) is received to a hydrothermal decomposition reactor. In embodiments, the hydrothermal decomposition reactor may include the hydrothermal decomposition reactor 304 as described in FIG. 3. At 906, Carbon Dioxide (CO₂) and Hydrogen (H₂) are produced in the hydrothermal decomposition reactor in accordance with methods described herein. For example, the reaction conditions within the production of Carbon Dioxide (CO₂) and Hydrogen (H₂) follows Equation 3 and occurs at an operating temperature in the range of about 200-400° C., a pressure of about 1-50 bar, and in the presence of a catalyst, such as a nickel (Ni) based catalyst, or another group 8 to 10 metal-based catalyst.

At 908, the Carbon Dioxide (CO₂) produced in 902 is received into a Solid Oxide Stack, such as the Solid Oxide Stack 402 described in FIG. 4. At 910, Carbon Monoxide (CO) is produced in the Solid Oxide Stack in accordance with methods described herein.

At 912, the Hydrogen (H₂) from 906 and the Carbon Monoxide (CO) from 910 are received in a methanol synthesis reactor, such as the Methanol Synthesis Reactor 502 described in FIG. 5. At 914, Methanol (CH₃OH) is produced in the methanol synthesis reactor in accordance with methods described herein.

### CONCLUSION

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Although the application describes embodiments having specific structural features and/or methodological acts, it is to be understood that the claims are not necessarily limited to the specific features or acts described. Rather, the specific features and acts are merely illustrative some embodiments that fall within the scope of the claims.

What is claimed is:

1. An Integrated Energy System (IES) comprising:
a power plant configured to generate steam,
a hydrothermal decomposition plant configured to receive Methane (CH₄) and at least a first portion of the steam, and to produce Carbon Dioxide (CO₂) and Hydrogen (H₂),
an electrolysis plant configured to receive the Carbon Dioxide (CO₂) and to produce Carbon Monoxide (CO), and
a methanol synthesis reactor configured to receive the Hydrogen (H₂) and the Carbon Monoxide (CO) to produce Methanol (CH₃OH).

2. The IES of claim 1, wherein the power plant comprises at least one nuclear reactor and electrical power generation system, the at least one nuclear reactor being configured to generate the steam, and the electrical power generation system being configured to generate electricity.

3. The IES of claim 2, wherein the hydrothermal decomposition plant receives at least a portion of the electricity from the power plant.

4. The IES of claim 1, wherein the hydrothermal decomposition plant comprises:
a reactor configured to receive the Methane (CH₄) and water (H₂O) to produce Carbon Dioxide (CO₂) and Hydrogen (H₂), and
wherein the first portion of the steam from the power plant provides thermal energy to the reactor.

5. The IES of claim 4, wherein the water (H₂O) comprises the first portion of the steam from the power plant.

6. The IES of claim 4, wherein the hydrothermal decomposition plant further comprises a first separation unit configured to separate the Carbon Dioxide (CO₂) and the Hydrogen (H₂).

7. The IES of claim 1, wherein the electrolysis plant comprises a Solid Oxide Stack configured to produce the Carbon Monoxide (CO) and Oxygen (O₂).

8. The IES of claim 7, wherein the electrolysis plant further comprises:
a first separation unit configured to separate the Carbon Dioxide (CO₂) from the Carbon Monoxide (CO) produced in the Solid Oxide Stack, and
a second separation unit configured to separate the Carbon Dioxide (CO₂) from the Oxygen (O₂) produced in the Solid Oxide Stack.

9. The IES of claim 2, wherein the electrolysis plant is configured to receive at least a portion of the electricity from the power plant.

10. The IES of claim 2, wherein the methanol synthesis reactor is configured to receive at least a portion of the electricity from the power plant.

11. An Integrated Energy System (IES) comprising:
a power plant configured to generate steam;
a hydrothermal decomposition reactor configured to receive at least a portion of the steam (H₂O) from the power plant to react with Methane (CH₄) within the hydrothermal decomposition reactor to produce Hydrogen (H₂) and Carbon Dioxide (CO₂);
a first separation unit configured to separate the Hydrogen (H₂) and the Carbon Dioxide (CO₂);
a Solid Oxide Stack configured to receive at least a portion of the Carbon Dioxide (CO₂) and to produce Carbon Monoxide (CO);
a second separation unit configured to separate the Carbon Dioxide (CO₂) from the Carbon Monoxide (CO); and
a methanol synthesis reactor configured to receive at least a portion of the Hydrogen (H₂) and at least a portion of the Carbon Monoxide (CO) to produce Methanol (CH₃OH).

12. The IES of claim 11, wherein the power plant comprises at least one nuclear reactor and electrical power generation system, the at least one nuclear reactor being configured to generate the steam, and the electrical power generation system being configured to generate electricity.

13. The IES of claim 11, wherein the hydrothermal decomposition reactor comprises a group 8 to 10 alloy catalyst and the methanol synthesis reactor comprises a Nickel Oxide (NiO₂) catalyst.

14. The IES of claim 12, wherein the hydrothermal decomposition reactor is configured to receive at least a first portion of the electricity from the power plant and the methanol synthesis reactor is configured to receive at least a second portion of the electricity from the power plant.

15. A method comprising:
receiving steam and electricity from a power plant to a hydrothermal decomposition reactor;
receiving Methane (CH₄) to the hydrothermal decomposition reactor to react with at least a portion of the steam to produce Carbon Dioxide (CO₂) and Hydrogen (H₂);
receiving the Carbon Dioxide (CO₂) into a Solid Oxide Stack to produce Carbon Monoxide (CO); and
receiving the Hydrogen (H₂) and the Carbon Monoxide (CO) to a methanol synthesis reactor configured to produce Methanol (CH₃OH).

16. The method of claim 15, wherein the power plant comprises at least one nuclear reactor and electrical power generation system, the at least one nuclear reactor being configured to generate steam, and the electrical power generation system being configured to generate the electricity.

17. The method of claim 15, further comprising maintaining the hydrothermal decomposition reactor at a temperature between 200-400° C. and a pressure between 1-50 bar.

18. The method of claim 15, further comprising receiving the steam and the Methane (CH₄) to the hydrothermal decomposition reactor at a ratio of 1:4.

19. The method of claim 15, wherein the hydrothermal decomposition reactor comprises at least one of a Nickle (Ni) based catalyst or a Ruthenium (Ru) based catalyst.

20. The method of claim 15, further comprising maintaining the methanol synthesis reactor at a temperature between 20° and 300° C. and a pressure between 50 and 100 bar.
